(19) United States
(12) Patent Application Publication
(10) Pub. No.: US 2012/0083747 A1
(43) Pub. Date: Apr. 5, 2012

(54) Vaginal renewal complex and applicator

(76) Inventors: Leo Galland, New York, NY (US); Olga Cohen, East Northport, NY (US)

(21) Appl. No.: 13/200,851

(22) Filed: Oct. 3, 2011

Related U.S. Application Data
(60) Provisional application No. 61/404,360, filed on Oct. 1, 2010.

(51) Int. Cl. A61K 36/185; A61P 15/02; A61M 5/31; C07C 57/12

(52) U.S. Cl. 604/218; 424/725; 554/224

(57) ABSTRACT

The invention provides a formulation designed to enhance a woman's genital health and comfort. The formulation is designed to be applied into the vaginal opening with an applicator. The formulation consists of five essential ingredients: evening primrose (Oenothera biennis) oil, shea (Butyrospermum parkii) oil, vitamin E (alpha-tocopherol), phenoxyethanol and vitamin D3 (cholecalciferol). These ingredients act synergistically to enhance the plasticity of the vaginal lining preventing and reversing dryness and discomfort. Each of these ingredients is fat soluble, and each is readily absorbed by the vaginal lining cells and synergistic action of these components occurs through creating changes in the cells, themselves, rather than merely coating the cells externally, as do vaginal lubricating gels.

### CLAIM OF PRIORITY

This patent application claims priority under 35 USC 119 (e) (1) from U.S. Provisional Patent Application Ser. No. 61/404,360, filed Oct. 1, 2010, of common inventorship herewith entitled, “Vaginal Renewal Complex and Applicator.”

### FIELD OF THE INVENTION

The present invention pertains to the field of natural formulations for vaginal comfort in post-menopausal women, and more specifically to the field of natural formulations for vaginal comfort in post-menopausal women administered in an applicator.

### BACKGROUND OF THE INVENTION

The prior art has put forth several formulations for treating vaginal dryness and other conditions. Among these are:

U.S. Pat. No. 6,893,648 to Harold Mermelstein and Frank P. Marchese is directed to a composition and method for treatment of vaginal dryness comprising a suppository containing herbal compounds with or without a vitamin such as vitamin A, vitamin C, vitamin D or vitamin E.

U.S. Patent Application number 2004/017010 to Nabil Sadek El Mogy is directed to the medical effect of jojoba oil in the treatment of vaginal diseases.

U.S. Pat. No. 4,765,978 to Syed E. Abidi is derected to a novel vaginal suppository comprising a pharmaceutical composition in a vegetable oil base.

None of these prior art references describe the present invention.

### SUMMARY OF THE INVENTION

It is an object of the present invention to provide a natural formulation for preventing vaginal dryness in post-menopausal women comprising gamma linolenic acid (GLA) found in evening primrose oil, shea oil, phenoxyethanol, vitamin E and vitamin D.

It is a further object of the present invention to provide the above formulation in a novel plunger type applicator.

### BRIEF DESCRIPTION OF THE DRAWINGS

FIG. 1 is a front perspective view of the applicator and cap of the present invention to comfortably administer the formulation of the present invention into the vaginal opening of a woman.

### DETAILED DESCRIPTION OF THE INVENTION

The present invention is a formulation designed to enhance a woman's genital health and comfort. The formulation is designed to be applied into the vaginal opening with an applicator. The formulation consists of five essential ingredients: evening primrose (Oenothera biennis) oil, shea (Butyrospermum parkii) oil, vitamin E (alpha-tocopherol), phenoxyethanol and vitamin D3 (cholecalciferol). These ingredients act synergistically to enhance the plasticity of the vaginal lining preventing and reversing dryness and discomfort. Each of these ingredients is fat soluble, and each is readily absorbed by the vaginal lining cells and synergistic action of these components occurs through creating changes in the cells, themselves, rather than merely coating the cells externally, as do vaginal lubricating gels.

The applicator is a plunger-type applicator with a specially designed contoured tip for comfortable insertion into the vaginal opening of a woman.

Evening primrose oil contains the unique fatty acid, gamma linolenic acid (GLA) which fights dryness, inflammation and hormonal imbalance and has been proven to have anti-inflammatory effects when administered to humans. The evening primrose oil in the present formulation improves the suppleness and lubrication of the vaginal lining. Taken orally, GLA has been shown to relieve some symptoms associated with dry eye syndrome, premenstrual syndrome, and menopause, and to improve the hydration of the barrier function of the skin. In the present formulation, GLA from evening primrose oil is directly absorbed from the vaginal cavity by cells lining the vaginal canal, where it exerts it beneficial physiological effects.

Shea oil is widely used in the cosmetic industry as a moisturizer and salve for the skin. It is a soothing natural moisturizer that contains substances with antioxidant effects. In the present formulation it is used to enhance the even spreading of evening primrose oil through the vaginal canal.

Vitamin E is an antioxidant that specifically protects polyunsaturated fatty acids, like GLA, from damage caused by oxidation. Used alone, vitamin E is a powerful antioxidant that improves healing of damages tissues. Vitamin E is used in the present formulation to protect the GLA and evening primrose oil, enhancing its effectiveness, and helping the GLA to stay active for a longer time.

Vitamin D is best known for its effects for improving the strength of bones, but recent research has demonstrated numerous other benefits, including immune enhancing, anti-viral and anti-cancer effects. The vaginal effects of vitamin D are especially important when estrogen levels are low, as occurs after menopause. A large percentage of North American women have low blood levels of Vitamin D. When taken orally, vitamin D has been shown to have diverse effects in women, including prevention of viral infection in winter, decreased risk of falls and fractures, improvement in muscle strength, decreased risk of post-menopausal breast cancer and improvement in mood and pain among people with overt vitamin D deficiency. As used in the present formulation, vitamin D is intended to enhance resistance of the vaginal lining to irritation and injury.

Phenoxyethanol serves as a bactericide and stabilizer.

The quantitative composition of the formulation of the present invention is as follows:
Evening Primrose Oil 92%
Shea Oil 5%
Vitamin E Acetate 1.0%
Vitamin D3 1.0%
Phenoxyethanol 1.0%.

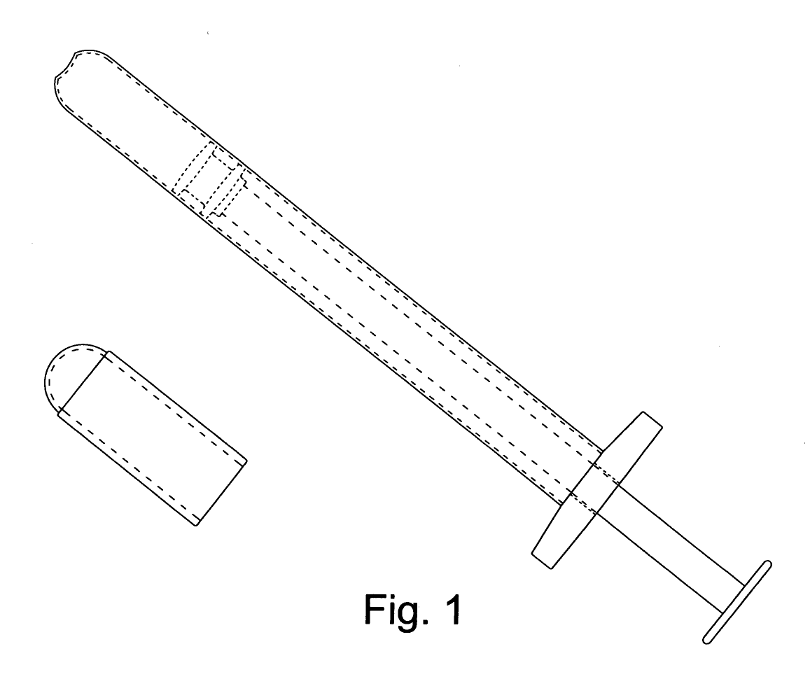

A further embodiment of the present invention is the applicator shown in FIG. 1. The applicator is a plunger-type applicator with a specially designed contoured tip for comfortable insertion into the vaginal opening of a woman.

Although this invention has been described with respect to specific embodiments, it is not intended to be limited thereto and various modifications which will become apparent to the person of ordinary skill in the art are intended to fall within the spirit and scope of the invention as described herein taken in conjunction with the accompanying drawings and the appended claims.

1. A natural formulation for preventing vaginal dryness in post-menopausal women comprising gamma linolenic acid (GLA) found in evening primrose oil, shea oil, phenoxyethanol, vitamin E and vitamin D.

2. The formulation of claim 1 comprising: 92% Evening Primrose Oil, 5% Shea Oil, 1% Vitamin E Acetate, 1.0%, Vitamin D3, and 1.0% Phenoxyethanol.

3. The formulation of claim 1 in a plunger type applicator having a contoured tip for comfortable insertion into the vaginal opening of a woman.

4. A natural formulation for preventing vaginal dryness in post-menopausal women comprising gamma linolenic acid (GLA) found in evening primrose oil.

5. The formulation of claim 3 in a plunger type applicator having a contoured tip for comfortable insertion into the vaginal opening of a woman.

6. An applicator comprising a plunger type applicator having a contoured tip for comfortable insertion into the vaginal opening of a woman.
